(19) United States
(12) Patent Application Publication
(10) Pub. No.: US 2024/0108912 A1
(43) Pub. Date: Apr. 4, 2024

(54) Methods of Using Light Energy to Facilitate Oxidative Phosphorylation

(71) Applicant: Erchonia Corporation, LLC, Melbourne, FL (US)

(72) Inventors: Steven C. SHANKS, Melbourne, FL (US); Travis SAMMONS, Melbourne, FL (US)

(73) Assignee: Erchonia Corporation, LLC, Melbourne, FL (US)

(21) Appl. No.: 18/239,012

(22) Filed: Aug. 28, 2023

Related U.S. Application Data
(60) Provisional application No. 63/413,153, filed on Oct. 4, 2022.

(51) Int. Cl. A61N 5/067

(52) U.S. Cl. CPC A61N 5/067; A61N 2005/0651

(57) ABSTRACT

Each electron transport chain complex has different wavelengths at which there is peak absorption of the light. Methods described herein apply light at defined ranges of wavelengths to a patient's skin, targeting each complex of the electron transport chain at its peak absorption, to increase the activity of the respective complex. This results in improved oxidative phosphorylation and mitochondrial function in the area treated. Complexes I and II are treated with violet-blue (400-490 nm) light. Complex I is treated with green (495-570 nm) light. Complex IV is treated with red (620-700 nm) light. To achieve optimal oxidative phosphorylation all three colors are applied to the treatment area, simultaneously or non-simultaneously. The light energy can be applied using a single device that can emit all three wavelengths, or multiple devices that emit a subset of the desired wavelengths.

### CROSS-REFERENCE TO RELATED APPLICATIONS

This application claims the benefit of co-pending U.S. Provisional Application No. 63/413,153 filed Oct. 4, 2022.

### FIELD OF INVENTION

This invention relates generally to modulating the electron transport chain using light energy. This invention relates particularly to upregulating the electron transport chain complexes with the non-invasive application of light energy.

### BACKGROUND

Oxidative phosphorylation is the metabolic pathway in which cells use enzymes to oxidize nutrients, thereby releasing chemical energy in order to produce metabolic energy in the form of adenosine triphosphate (ATP). ATP is the energy source in all biological systems including skeletal, muscular, nervous, endocrine, cardiovascular, lymphatic, respiratory, digestive, urinary, and the reproductive system. ATP hydrolysis provides the energy needed for many essential processes in organisms and cells. These processes include intracellular signaling, DNA and RNA synthesis, purinergic signaling, synaptic signaling, active transport, and muscle contraction. In animals, plants, fungi and other eukaryotes, oxidative phosphorylation takes place inside mitochondria. Typically, mitochondria supply more than 90% of a cell's ATP requirement. Non-invasive methods known in the art permit in vivo assessment of mitochondrial function, such as those based on magnetic resonance spectroscopy (MRS) and near-infrared spectroscopy (NIRS). Moreover, it is now possible to gather information on regulation of mitochondrial content by measuring the in vivo synthesis rate of individual mitochondrial proteins.

Oxidative phosphorylation has two parts: the electron transport chain and chemiosmosis. The electron transport chain is series of protein complexes and other molecules bound to the inner mitochondrial membrane which transfer electrons from electron donors to electron acceptors via redox reactions. The electron transport chain proteins in a general order are Complex I, Complex II, coenzyme Q, Complex III, cytochrome C, and Complex IV. The energy transferred by electrons flowing through this electron transport chain is used to transport protons across the inner mitochondrial membrane. This generates potential energy in the form of a pH gradient and an electrical potential across this membrane. This store of energy is tapped when protons flow back across the membrane and down the potential energy gradient, through a large enzyme called ATP synthase in a process called chemiosmosis.

Each complex of the electron transport chain plays a crucial role in oxidative phosphorylation. If one or more of the complexes is inhibited, oxidative phosphorylation will be limited, due to an inhibition of flow of electrons through the other complexes resulting in less proton pumping, ATP synthesis, and oxygen uptake. Inhibition of one or more complexes has been linked to disease and disorders. In one study comparing obese women to non-obese women, a higher BMI correlated to significantly fewer complex I and IV components in adipose tissues. This suggests that obesity is associated with impaired mitochondrial function in adipocytes. In a separate study, impaired oxidative phosphorylation is suggested as a factor behind insulin resistance of skeletal muscle in type 2 diabetes.

Several mitochondrial defects manifesting as decreased activity of electron transport chain complexes and high reactive oxygen species levels have been identified in central nervous system disorders.

Membrane leakage and electrolyte imbalances, pro-apoptotic pathway activation, and mitophagy are among the mechanisms implicated in the pathogenesis of neurodegenerative diseases, such as Alzheimer's, Parkinson's, and Huntington's disease, as well as ischemic stroke. In a separate study, significantly lower levels of complexes III and V were found in the cerebellum of autistic children compared to age-matched control children.

To produce optimal biological results, each complex in the electron transport chain needs to be operating efficiently. It would be desirable to have a way to facilitate oxidative phosphorylation by increasing the activity and functioning ability of electron transport chain complexes.

Low-level laser therapy (“LLLT”) has been shown through numerous clinical studies and regulatory clearances to be a safe and effective, simple, non-invasive and side-effect free alternative to medication and surgical procedures for the reduction of symptoms in a variety of conditions. LLLT reduces edema, improves wound healing, and relieves pain of various etiologies. It is also used in the treatment and repair of injured muscles and tendons. Application of LLLT has been shown to have the potential to alter cellular metabolism to produce a beneficial clinical effect.

Based on its ability to modulate cellular metabolism and alter the transcription factors responsible for gene expression, LLLT has been found to alter gene expression, cellular proliferation, intra-cellular pH balance, mitochondrial membrane potential, generation of transient reactive oxygen species and calcium ion level, proton gradient and consumption of oxygen. LLLT stimulation of the mitochondria via low-energy light has been shown to provoke a dynamic shift in the function of an individual cell. Laser therapy has been shown to stimulate cell regeneration and later gene expression.

To date, research and clinical applications of light therapy have been primarily limited to the targeting cytochrome c oxidase or Complex IV through the absorption of red or near-infrared light. It would be desirable to have a way to selectively target the other electron transport chain molecules, and particularly the complexes to increase the activity and functioning ability of electron transport chain complexes.

### SUMMARY OF THE INVENTION

Each electron transport chain complex has different wavelengths at which there is peak absorption of the light. Methods described herein apply light at defined ranges of wavelengths to a patient's skin, targeting each complex of the electron transport chain at its peak absorption, to increase the activity of the respective complex. This results in improved oxidative phosphorylation and mitochondrial function in the area treated. Complexes I and II are treated with violet-blue (400-490 nm) light. Complex I is treated with green (495-570 nm) light. Complex IV is treated with red (620-700 nm) light. To achieve optimal oxidative phosphorylation all three colors are applied to the treatment area, simultaneously or non-simultaneously. The light energy can be applied using a single device that can emit all three wavelengths, or multiple devices that emit a subset of the desired wavelengths.

### BRIEF DESCRIPTION OF THE DRAWINGS

FIG. 1 illustrates the electron transport chain and the application of colored light to specific complexes.

FIG. 2 illustrates three colors of light passing through a lens resulting in white light.

FIG. 3 is a schematic illustration of a brain inside a patient's skull.

FIG. 4 is a schematic illustration of a brain.

### DETAILED DESCRIPTION OF THE INVENTION

Methods described herein utilize light at defined ranges of wavelengths to target each complex of the electron transport chain at its peak absorption to increase the activity of each complex. This improves oxidative phosphorylation and mitochondrial function. Complex I and II are treated with violet-blue (400-490 nm) light. Complex III is treated with green (495-570 nm) light. Complex IV is treated with red (620-700 nm) light. To achieve optimal oxidative phosphorylation all three colors are applied to the treatment area, simultaneously or non-simultaneously.

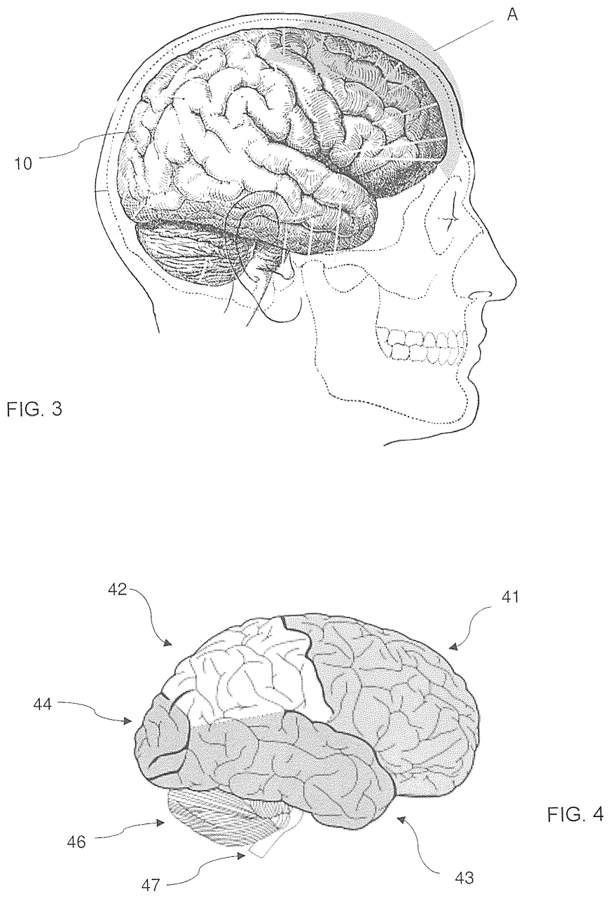

The methods are non-invasive. The light is applied externally to a patient's skin at or near the area of the malady to be treated. This area of skin to be treated is referred to herein as the treatment area. As used herein, light applied “near the” area means light applied to the skin of the patient where the wound, pain, disorder or disease is occurring. For example, to treat a neurodegenerative disorder, the light energy is applied to the scalp at a position mapped to the area of the brain 10 to be treated, such as the frontal 41, parietal 42, temporal 43, and occipital 44 lobes; the cortex; cerebellum 46; the brain stem 47 or base of the brain; or where one or more cranial nerves enters the brain. See FIGS. 3 and 4. For example, if the light is to be applied “near the frontal lobes,” it will be applied to the scalp above the frontal cortex, as indicated generally by area A in FIG. 3. In another example, if the treatment is to be applied to a torn ACL, the light energy is applied at or near the knee.

There are a number of variables in light therapy beyond the wavelength of the light, including the power of the light source, the area impinged by the light, the shape of the beam spot when the light impinges the treated area, the pulse frequency, the intensity or fluence of the light energy, and the treatment duration. The setting of these variables typically depends heavily on the disease and tissue characteristics of the specific patient. The success of each therapy depends on the relationship and combination of these variables. For example, as disclosed in more detail below, Alzheimer's disease may be treated with one regimen utilizing a given power, wavelength, pulse frequency and treatment duration, whereas autism may be treated with a regimen utilizing a different power, wavelength, pulse frequency and treatment duration, and either regimen may be further adjusted for a given patient depending on that patient's size, weight, age, and stage of the disease.

The light can be from any source including light-emitting diodes, hard-wired lasers, or laser diodes, but preferably is from semiconductor laser diodes. Commercial semiconductor laser diodes have a spread of ±10 nm from nominal so, for a given desired wavelength, the light applied is within the spread from nominal.

The applied light is typically from emitters of less than 1W. This low-level light therapy has an energy dose rate that causes no immediate or long-term detectable temperature rise of the treated tissue and no macroscopically visible changes in tissue structure. Consequently, the tissue impinged by the light is not heated and is not damaged. Because the tissue impinged by the light is not heated, no mechanism to cool the skin is needed. The light is applied directly to the tissue in the treatment area with no intervening temperature-reducing elements between the light-emitting device and the treatment area. The applied light energy may be applied continuously or applied with a pulse frequency or frequencies.

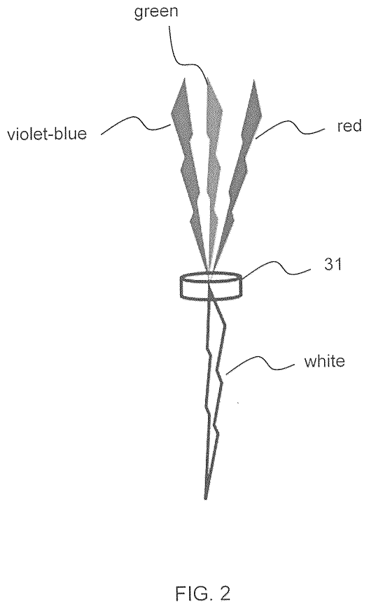

The light energy can be applied using a single device that can emit all three wavelengths that impinge the skin at different locations so that all three colors can be seen on the skin at the same time. Alternatively, light energy can be applied using a single device that has a lens 31 such as a set of one or more prisms that all three colors pass through simultaneously, resulting in a white light on the skin of the patent. See FIG. 2. Instead of a single device, light energy can be applied using multiple devices each of which emits a subset of the desired wavelengths. For example, two laser devices may be used wherein the first device emits red light and the second emits violet-blue and green.

In one embodiment, the light is emitted in a line and the line is passed across a person's skin in the treatment area. In a preferred embodiment, the line of laser light is emitted from a hand-held laser device and manually swept across the patient's skin in a continuous, sweeping motion. In another embodiment, the patient lies on a table and a laser light source passes over the patient. U.S. Pat. No. 8,439,959 incorporated herein by reference, discloses such a device to sweep laser energy across a patient.

In another embodiment the shape of the beam spot on the treated area is an apparent circle, which is actually a rotating diameter by a line of light. U.S. Pat. No. 7,922,751, incorporated herein by reference, discloses a device to sweep such a circular beam spot. The device disclosed in that patent can be programmed to move the scanning head in a manner to achieve any desired shape of a treatment zone on the patient. A sample selection of available scan patterns is shown in that patent at FIGS. 8a-h.

The three defined colors of light, violet-blue, green, and red, can be applied in several methods. One approach applies light energy of each of the three colors sequentially, activating one or two electron transport chain complexes with each color. Six sequences are available, each a method:

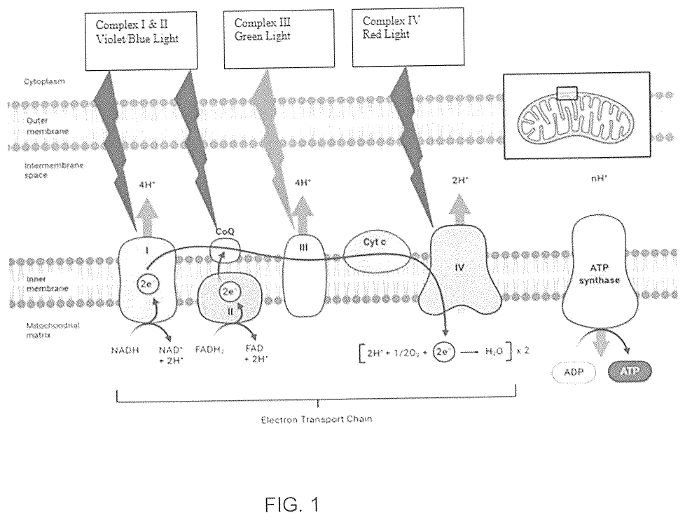

In one embodiment the activity of Complex I is increased by applying light energy having a violet-blue wavelength to a treatment area of a patient's skin; the activity of Complex II is increased by applying light energy having a violet-blue wavelength to the treatment area; the activity of Complex III is increased by applying light energy having a green wavelength to the treatment area; and the activity of Complex IV is increased by applying light energy having a red wavelength to the treatment area. See FIG. 1.

In another embodiment the light energy is applied in order of green, violet-blue, and red, increasing the activity of Complex III, Complex I and Complex II, and Complex IV, respectively.

In another embodiment the light energy is applied in order of green, red and violet-blue, increasing the activity of Complex III, Complex IV, Complex I and Complex II, respectively.

In another embodiment the light energy is applied in order of red, violet-blue, and green, increasing the activity of Complex IV, Complex I and Complex II, and Complex III, respectively.

In another embodiment the light energy is applied in order of violet-blue, red, and green, increasing the activity of Complex I and Complex II, Complex IV, and Complex III, respectively.

In another embodiment the light energy is applied in order of red, green, and violet-blue, increasing the activity of Complex IV, Complex III, and Complex I and Complex II, respectively.

Yet another method directs red, green, and violet-blue light through a lens 31, resulting in the emission of a white beam of light. While the beam of light appears white, the three wavelength properties are maintained. See FIG. 2. The activity of Complex I and Complex II, Complex III, and Complex IV is increased simultaneously, and there may be a cascade effect in which increased activity of Complex I itself increases the activity of Complex II, which in turn increases the activity of Complex III, which increases the activity of Complex IV.

Treatment examples for treating neck and shoulder pain:
Example 1: Light is applied to the deltoid and trapezius muscles in the following order: 5 minutes 405 nm violet light, followed by 5 minutes of 520 nm green light, and lastly 5 minutes 635 nm red light.
Example 2: Light is applied simultaneously at three separate wavelengths from different emitters: one emitting at 450 nm, one emitting at 532 nm, and one emitting 650 nm to the trapezius muscles for 10 minutes, followed by 10 minutes of exposure of the 450 nm, 532 nm, and 650 nm light energy to the deltoid.
Example 3: A device that directs red, green, violet light into one lens emits a white laser beam that is applied to the trapezius for 10 minutes, followed by 10 minutes of exposure to the deltoid.

Treatment examples for non-invasive fat loss:
Example 4: Light energy is applied to the waist, hips, and thighs in the following order: 10 minutes at 540 nm green, followed by 10 minutes of 415 nm violet light, and lastly 10 minutes of 660 nm red light.
Example 5: Light is applied simultaneously at three separate wavelengths from different emitters: one emitting at 415 nm, one emitting at 560 nm, and one emitting at 620 nm applied to the waist, hips, and thighs for 40 minutes.
Example 6: A device that directs red, green, violet light into one lens emits a white laser beam that is applied to the waist, hips, and thighs for 40 minutes.

Treatment examples for autism:
Example 7: Light energy is applied to the frontal cortex and temporal lobe in the following order: 5 minutes at 415 nm violet light, followed by 5 minutes of 520 nm green light, and lastly 5 minutes of 635 nm red light.
Example 8: Light is applied simultaneously at three separate wavelengths from different emitters: one emitting at 415 nm, one emitting at 520 nm, and one emitting at 650 nm applied to the frontal lobe for 10 minutes, followed by 10 minutes of applying light energy at 450 nm, 532 nm, and 650 nm to the temporal lobes.
Example 9: A device that directs red, green, violet light into one lens emits a white laser beam that is applied to the scalp near the frontal lobe for 5 minutes, followed by 5 minutes of exposure to the scalp near the temporal lobes.

In some embodiments, the mitochondrial function of a patient is measured in vivo before treatment with light energy. The light energy is then applied at one or more wavelengths to increase the activity of one or more complexes in the electronic transport chain thus improving oxidative phosphorylation and mitochondrial function. After the light energy is applied, the mitochondrial function of a patient is measured again in vivo. Additional treatments of light energy at one or more wavelengths may be applied until the mitochondrial function is at a desired level.

While there has been illustrated and described what is at present considered to be the preferred embodiments of the present invention, it will be understood by those skilled in the art that various changes and modifications may be made and equivalents may be substituted for elements thereof without departing from the true scope of the invention. Therefore, it is intended that this invention not be limited to the particular embodiments disclosed, but that the invention will include all embodiments falling within the scope of the appended claims.

1. A method for increasing activity of one or more complexes in the electron transport chain in a treatment area of a patient, the method comprising:
a. increasing the activity of Complex I by applying light energy having a violet-blue wavelength externally to the treatment area of a patient;
b. increasing the activity of Complex II by applying light energy having a violet-blue wavelength externally to the treatment area;
c. increasing the activity of Complex III by applying light energy having a green wavelength externally to the treatment area; or
d. increasing the activity of Complex IV by applying light energy having a red wavelength externally to the treatment area.

2. The method of claim 1 wherein the light energy is low-level laser energy applied to the treatment area in a continuous sweeping motion.

3. The method of claim 2 wherein the application of laser energy causes no detectable temperature rise of the treated location.

4. The method of claim 2 wherein low-level laser energy is applied using a hand-held laser device that emits a line of laser light.

5. The method of claim 1 wherein the violet-blue, green, and red wavelengths are emitted from a single light-emitting device.

6. The method of claim 1 wherein more than one of the wavelengths are applied simultaneously.

7. A method for increasing activity of one or more complexes in the electron transport chain in a treatment area of a patient, the method comprising applying light energy of one or more of the following wavelengths externally to the treatment area:
a. light energy having a violet-blue wavelength;
b. light energy having a green wavelength; and
c. light energy having a red wavelength;
wherein
d. light energy having a violet-blue wavelength increases activity of Complex I or Complex II;
e. light energy having a green wavelength increases activity of Complex III; and
f. light energy having a red wavelength increases activity of Complex IV.

8. The method of claim 7 wherein the light energy is applied in the following order:
a. light energy having a violet-blue wavelength;
b. light energy having a green wavelength; and
c. light energy having a red wavelength.

9. The method of claim 7 wherein the light energy is applied in the following order:
a. light energy having a green wavelength;
b. light energy having a violet-blue wavelength; and
c. light energy having a red wavelength.

10. The method of claim 7 wherein the light energy is applied in the following order:
a. light energy having a green wavelength;
b. light energy having a red wavelength; and
c. light energy having a violet-blue wavelength.

11. The method of claim 7 wherein the light energy is applied in the following order:
a. light energy having a red wavelength;
b. light energy having a violet-blue wavelength; and
c. light energy having a green wavelength;

12. The method of claim 7 wherein the light energy is applied in the following order:
a. light energy having a violet-blue wavelength;
b. light energy having a red wavelength; and
c. light energy having a green wavelength.

13. The method of claim 7 wherein the light energy is applied in the following order:
a. light energy having a red wavelength;
b. light energy having a green wavelength; and
c. light energy having a violet-blue wavelength.

14. The method of claim 7 wherein the violet-blue, green, and red wavelengths are applied simultaneously.

15. The method of claim 7 wherein the light energy is low-level laser energy applied to the treatment area in a continuous sweeping motion.

16. The method of claim 15 wherein the application of laser energy causes no detectable temperature rise of the treated location.

17. The method of claim 15 wherein low-level laser energy is applied using a hand-held laser device that emits a line of laser light.

18. The method of claim 7 wherein a single light-emitting device applies the violet-blue, green, and red wavelengths.

19. A method for increasing the mitochondrial function in a treatment area of a patient, the method comprising:
a. measuring the mitochondrial function of a patient;
b. applying a first treatment of light energy at one or more wavelengths, the one or more wavelengths increasing the activity of one or more complexes in the electronic transport chain;
c. measuring the mitochondrial function of a patient after applying light energy; and
d. applying a second treatment of light energy at the one or more wavelengths, the one or more wavelengths increasing the activity of one or more complexes in the electronic transport chain until the mitochondrial function is at a desired level.

20. The method of claim 19 wherein the one or more wavelengths are selected from violet-blue, green, and red wavelengths.

21. The method of claim 19 wherein the light energy is low-level laser energy applied to the treatment area in a continuous sweeping motion.

22. The method of claim 21 wherein the application of laser energy causes no detectable temperature rise of the treated location.

23. The method of claim 21 wherein low-level laser energy is applied using a hand-held laser device that emits a line of laser light.
